SEQUENCE LISTING (6 sequences)

SEQ ID NO: 1 - protein, 273 aa, Homo sapiens
MRGSQEVLLMWLLVLAVGGTEHAYRPGRRVCAVRAHGDPVSESFVQRVYQPFLTTCDGHRACSTYRTIYRTAYRRSPGLAPARPRYACCPGWKRTSGLPGACGAAICQPPCRNGGSCVQPGRCRCPAGWRGDTCQSDVDECSARRGGCPQRCVNTAGSYWCQCWEGHSLSADGTLCVPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS

SEQ ID NO: 2 - protein, 288 aa, Homo sapiens (Human)
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 195 aa, Homo sapiens (Human)
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 4 - protein, 241 aa, Homo sapiens (Human)
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 5 - protein, 390 aa, Homo sapiens (Human)
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 6 - protein, 232 aa, Homo sapiens (Human)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

(19) United States
(12) Patent Application Publication
(10) Pub. No.: US 2017/0266354 A1
(43) Pub. Date: Sep. 21, 2017

(54) Cell-Based Device For Local Treatment With Therapeutic Protein

(71) Applicant: Kemijski Institut, Ljubljana (SI)

(72) Inventors: Lucija KADUNC, Bled (SI); Dusko LAINSCEK, Ljubljana (SI); Simon HORVAT, Preddvor (SI); Roman JERALA, Ljubljana (SI); Iva HAFNER BRATKOVIC, Ljubljana (SI)

(21) Appl. No.: 15/508,548

(22) PCT Filed: Sep. 4, 2014
(86) PCT No.: PCT/SI2014/000050

§ 371 Date: Mar. 3, 2017

(51) Int. Cl. A61L 31/16; A61K 38/18; A61L 31/00

(52) U.S. Cl. CPC A61L 31/16; A61L 31/005; A61K 38/1808; A61K 38/1825; A61K 38/18; A61K 38/1858; A61K 38/1841; A61K 38/1866; A61L 2300/414; A61L 2430/02

(57) ABSTRACT

The present invention provides a therapeutic device that comprises of mixture of cells secreting combination of therapeutic proteins, where cells producing therapeutic proteins are sealed in container which enables the exchange of nutrient and therapeutic proteins. The cells inside the therapeutic device produce and secrete certain amounts of therapeutic proteins. Cells are prepared by introducing genes encoding therapeutic proteins under the control of a constitutive or inducible promoter. The combination and concentration of therapeutic proteins is defined by the ratio of cells secreting different therapeutic proteins and/or by the gene expression ratio of the therapeutic proteins in the cells incorporated into the semi-permeable container. The therapeutic device can be used for treatments of various diseases and injuries for instance enhancement of wound healing and angiogenesis.

### FIELD OF THE INVENTION

The field of invention is directed at a therapeutic device comprising cells secreting a combination of therapeutic proteins, wherein the cells preferably are located in a container which enables the exchange of nutrients and therapeutic proteins.

The cells producing therapeutic proteins are present in the device in ratios and/or show expression and secretion of the therapeutic proteins which are appropriate for therapeutic application.

The present invention provides a device comprising cells secreting a combination of therapeutic proteins, and the use of said device for the treatment of diseases and injuries in which said therapeutic proteins are effective.

### BACKGROUND OF THE INVENTION

Individual growth factors have been tested for treatment of various diseases and injuries of humans and animals. PDGF-BB is useful in the treatment of diabetic foot ulcers, GM-CSF in the treatment of venous and diabetic foot ulcers and HGH in the treatment of pediatric burns. However, some individual soluble factors failed to improve medical condition of patients such as the combination therapy of IGF-1 and PDGF for treatment of diabetic foot ulcers or even had severe adverse effects, for example CNTF in treatment of ALS. In these trials soluble factors were applied at very high doses, since some have a short life-time in a living organism. Additionally, in the natural regenerative processes or in therapy by stem cells a combination of trophic factors is secreted providing the best therapy, which could not be reproduced by the use of a single factor. Many therapeutic activities of stem cells are due to the in situ production and their local delivery of trophic factors. In the past decade, many studies on animal models or even human trials used stem cells to treat diabetic wounds. When endothelial progenitor cells (EPCs) were used, the majority of studies proposed that EPCs exert the major healing effect via the paracrine action. Secretome of EPCs contains a variety of soluble factors, which promote wound healing via proliferation, migration and cell survival such as VEGF, PDGF, monocyte chemoattractant protein-1 and stromal-derived factor-1α (Barcelos et al., 2009; Di Santo et al., 2009; Zhang et al., 2009). Similar observations were made with mesenchymal stem cells (Javazon et al., 2007), which were shown to secrete VEGF, IGF-1, EGF, KGF, angiopoietin-1, stromal derived factor-1, MIF-1, erythropoietins (Wu et al., 2007). WO 2011/123779, US 2011/0020291 etc. disclose the use of stem cells for wound healing, however, there are some mayor issues connected to stem cells regarding the safety, cellular retention and high preparation costs.

U.S. Pat. No. 5,487,889 describes a bandage containing a container with cells, which are engineered to secrete human PDGF, human EGF, human TGF, bovine GH and combinations thereof to improve wound healing, yet some of those factors have already been shown not to be successful in clinical trials and additional trophic factors seem to be required for the effective therapy. Additionally, the use of bandage of U.S. Pat. No. 5,487,889 is limited to topical applications. Normal wound healing process is a complex cascade of interactions between different cell types, growth factors, matrix proteins and other bioactive proteins. However, in many human diseases, conditions and as a consequence of treatments the normal wound healing process is disrupted resulting in a chronic wound. Some of the conditions underlying abnormal wound healing are diabetes, chronic kidney disease, anemia, age, and malnutrition. Advanced treatments of such wounds include hyperbaric oxygen therapy, advanced wound dressings, growth factor treatment such as the use of Regranex (PDGF) and application of designed cellular products such as Apligraft and Dermagraft.

Current experimental therapeutic settings are based on isolated bioactive molecules or engineered cells secreting a biological drug of choice focus on a single soluble factor. Although such treatment was effective in animal models of neurodegeneration, it did not work when it was tested in clinical trials. Immune rejection of the implant, inappropriate route of administration and dosage used are most commonly mentioned as the reason for non-significant improvement or even termination of the trial due to severe negative side effects (Barinaga, 1994; Emerich et al., 2013; Zanin et al., 2012).

When working with genetically modified cells or isolated proteins, only a few studies actually looked at synergistic effects of two trophic factors. PDGF was shown to work synergistically with IGF-I or EGF in wound healing (Lynch et al., 1987). GDNF exhibits its full neurotrophic potential when TGF-β is present (Krieglstein et al., 1998). In the case of NT3 and NGF synergistic effect on the survival of cholinergic neurons in saporin treated rats was not observed (Lee et al., 2013). Stem cells on the other hand secrete a plethora of therapeutic proteins, which could act additively or synergistically. In mentioned studies, where they showed synergistic effect, they used high dosage of recombinant protein, the dosage, which is unphysiological and therefore could present a possible risk for tissue hyperproliferation and also tumor development. The mentioned studies did not show therapeutic value for regulated combination of proteins. With the fact that chronic wound treatment costs nearly 2% of European health budgets, we would like to stress the need for development of novel treatments for healing of chronic wounds and similar conditions.

Ischemic cardiovascular and cerebrovascular disorders originating from atherosclerosis-associated arterial infarction are leading causes for mortality in the Western society. The major problems in medicine also represent treatment of chronic ischemic wounds that occur due to the diabetes type I or II, neuropathy etc. The major causes of death in patients with diabetes are cardiovascular diseases, 52% respectively. Diabetes is associated with micro- and macrovascular complications, which result in heart conditions and ischemic infarction (heart, brain, and kidney). Diabetic patients are at high risk of developing peripheral arterial disease (PDA) and critical limb ischemia (CLI). Problem of normal blood flow occurs in all sorts of diseases, like in Berger's disease, coronary heart disease, ischemic infarction, that all can result in death.

### SUMMARY OF THE INVENTION

The present invention provides a therapeutic device releasing a combination of therapeutic proteins. The device comprises cells which simultaneously secrete a combination (i.e. a mixture) of therapeutic proteins, wherein said cells are preferably located in a container which enables the exchange of nutrients and therapeutic proteins.

Each cell type used in the present invention produces a certain amount of one or more specific therapeutic protein and is preferably prepared by the introduction of genes encoding therapeutic proteins under the control of (i.e. operatively linked to) a constitutive or inducible promoter into a carrier cell. The combination and concentration of therapeutic proteins is defined by the ratio of cells secreting different therapeutic proteins and/or by the gene expression ratio of the therapeutic proteins in the cells incorporated into the semi-permeable container.

The use of said therapeutic device, methods for application of said therapeutic device and the use of the therapeutic device for enhancement of wound healing and angiogenesis are also provided.

### BRIEF DESCRIPTION OF THE DRAWINGS

FIG. 1: A combination of therapeutic proteins promotes wound closure in a gap closure migration assay A day before experiment NIH 3T3 cells (5*10⁴ cells/well) were seeded onto 8 well μ-slide with inserted culture insert (Ibidi). Supernatants containing single therapeutic protein (EGFL7, FGF-2, IGF-I, PDGF-B, TGF-β1 or VEGF-A) or their combination were added to the cells. Cells were visualized 24 hours later (see Example 3). Percent (%) of gap closure was calculated for all conditions. An increased gap closure was observed in case of a combination of therapeutic proteins when compared to single therapeutic proteins or control without added therapeutic proteins.

FIG. 2: A combination of therapeutic proteins promotes wound closure in organ skin slices Skin punch biopsy samples were wounded using a scalpel. Supernatants of a combination of therapeutic cells producing a combination of therapeutic proteins (EGFL7, FGF-2, IGF-I, PDGF-B, TGF-β1 and VEGF-A) were added daily for 7 days (see Example 4). Afterwards skin was fixed using Histofix preservative for histology assessment. A standard HE-staining was performed. Histological analysis confirmed that in the presence of the combination of therapeutic proteins (B) significant improvement of wound closure was observed compared to non-treated control (A). In the skin slice treated with a combination of therapeutic proteins (B) there is smaller epithelial gap between wound margins due to the accumulation of macrophages, fibroblasts and fibrin production and the wound is almost completely closed in contrast to the control skin slice (A), in which there is no wound closure and there is greater epithelial gap then in treated skin slice.

FIG. 3: Therapeutic proteins are released from therapeutic cells restricted in a semipermeable container Mixture of therapeutic cells stably transfected with therapeutic proteins as described in example 5 was sealed in a semipermeable container and cultured in 6 well plate with 2 ml of DMEM/F-12 (5% FBS). Medium was sampled and replaced daily. Cells were cultivated for 14 days (Example 5). Concentration of secreted growth factors was measured using ELISA. Results show that encapsulated therapeutic cells produce and secrete growth factors into culture medium for at least 14 days.

FIG. 4: A therapeutic device, containing cells engineered to secrete therapeutic proteins, promotes wound healing in vivo Mice were wounded with 8 mm punch biopsy scalpel and afterwards the wound splints were stitched to the back of the mice, using medical cyanoacrylate glue and non absorbable sutures. Therapeutic device secreting therapeutic proteins (EGFL7, FGF-2, IGF-I, PDGF-B, TGF-β1 and VEGF-A) was placed in the centre of the wound. Surgical wound was covered and protected with adhesive sterile dressing. Wound healing was monitored daily via measurement and via taking photos. After 7 days, mice were humanely euthanized and skin samples, containing surgical wounds, were fixed for later histology and IHC assessment. In presence of therapeutic device (FIG. 4C) wound healing was significantly improved compared to when treated with device containing only carrier cells (FIG. 4B) or non-treated control (FIG. 4A). The wound closure due to the formation of neoepithelium is greater in C then in A and B. The wound area in animal with cellular device was smaller than in non-treated and in carrier cell line treated animal due to the formation of newly connective tissue (larger amounts of fibrin production and accumulation and therefore better wound healing). The HE staining confirmed the clinical results in animals. The skin samples from C showed completely closed wound due to the fibrin and reticulus fiber accumulation and cell infiltration (macrophages and fibroblasts). In the samples from A in B there is lesser wound closure, that results from greater epithelial gap between wound margins.

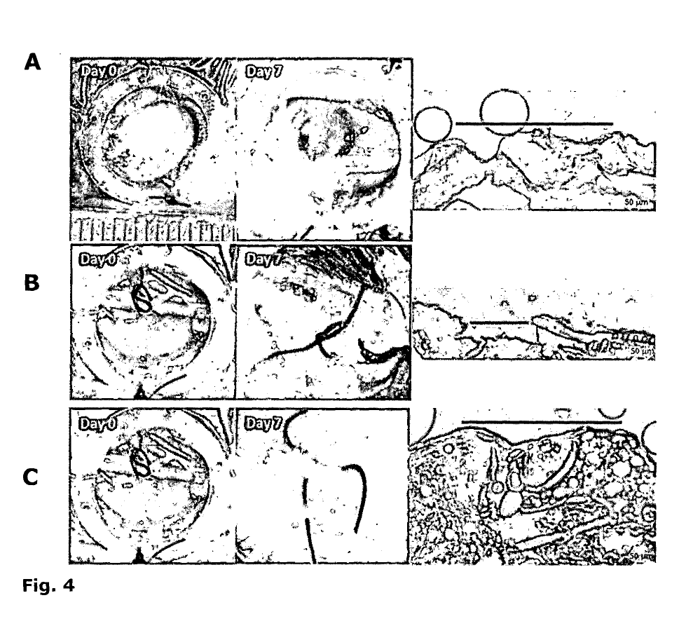

FIG. 5: A therapeutic device, containing cells engineered to secrete therapeutic proteins, promotes angiogenesis in vivo The effect of cellular device on postnatal arteriogenesis and angiogenesis was established using a mouse model of unilateral hind-limb ischemia, which is based on ligation and excising the femoral artery. Mice C57BL/6, aged 10-weeks were used. Near the ischemic region cellular device was implemented. In the control group of mice there was just unilateral hind-limb ischemia performed with no cellular device implementation. Mice were monitored daily. After the 7 days, mice were humanely euthanized and the gastrocnemius muscle was harvested. The hind-limb from the mouse with implanted cellular (right hind-limb on the picture) device showed less swelling and sub dermal edema then mouse with no cellular device (left hind-limb on the picture).

### DETAILED DESCRIPTION OF THE INVENTION

Before further description it is to be assumed that the invention is not limited to presented embodiments since modifications of particular embodiments can still be in the scope of appended claims. The terminology to be used in the description of the invention has the purpose of description of a particular segment of the invention and has no intention of limiting the invention. All publications mentioned in the description of the invention are listed as references. In the description of the invention and in the claims, the description is in the singular form, but also includes the plural form or vice versa, what is not specifically highlighted for the ease of understanding.

### Definitions

Unless defined otherwise, all technical and scientific terms used herein possess the same meaning as it is commonly known to experts in the field of invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following definitions of the several terms of the invention are provided. In each instance of their use in the remainder of the specification, these terms will have the respectively defined meaning and preferred meanings.

The term “therapeutic cells” or “therapeutic cell lines” refers to any kind of cells or cell lines being capable of expressing and secreting (mature) therapeutic proteins in their respective environment.

The term “carrier cell” or “carrier cell line” refers to cell or cell line from which therapeutic cell lines originate.

The term “implantable” having the possibility to be implanted into the body or on the surface of the body (e.g. implanted into a topical wound).

The term “therapeutic protein” refers to any kind of protein or polypeptide exerting a therapeutic action in a patient or animal.

The terms “polypeptide” and “protein” are used interchangeably herein and mean any peptide-linked chain of amino acids, regardless of length or post-translational modification.

As used herein, the term protein “variant” is to be understood as a polypeptide which differs in comparison to the polypeptide from which it is derived by one or more changes in the amino acid sequence. The polypeptide from which a variant is derived is also known as the parent polypeptide. Typically a variant is constructed artificially, preferably by recombinant DNA technology means. Typically, the polypeptide from which the variant is derived is a wild-type protein or wild-type protein domain. However, the variants usable in the present invention may also be derived from homologs, orthologs, or paralogs of the parent polypeptide or from artificially constructed variants, provided that the variant exhibits at least one biological activity of the parent polypeptide. The changes in the amino acid sequence may be amino acid exchanges, insertions, deletions, N-terminal truncations, or C-terminal truncations, or any combination of these changes, which may occur at one or several sites. In preferred embodiments, a variant usable in the present invention exhibits a total number of up to 30% (up to 1%, 2%, 3%, 4%, 5%, 6%, 7%, 8%, 9%, 10%, 15%, 20%, 25%, 30%) changes in the amino acid sequence (i.e. exchanges, insertions, deletions, N-terminal truncations, and/or C-terminal truncations). The amino acid exchanges may be conservative and/or non-conservative. In preferred embodiments, a variant usable in the present invention differs from the protein or domain from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acid exchanges, preferably conservative amino acid changes. Variants may additionally or alternatively comprise deletions of amino acids, which may be N-terminal truncations, C-terminal truncations or internal deletions or any combination of these. Such variants comprising N-terminal truncations, C-terminal truncations and/or internal deletions are referred to as “deletion variants” or “fragments” in the context of the present application. The terms “deletion variant” and “fragment” are used interchangeably herein. A deletion variant may be naturally occurring (e.g. splice variants) or it may be constructed artificially, preferably by gene-technological means. Typically, the protein or protein domain from which the deletion variant is derived is a wild-type protein. However, the deletion variants of the present invention may also be derived from homologs, orthologs, or paralogs of the parent polypeptide or from artificially constructed variants, provided that the deletion variants exhibit at least one biological activity of the parent polypeptide. Preferably, a deletion variant (or fragment) has a deletion of up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acids at its N-terminus and/or at its C-terminus and/or internally as compared to the parent polypeptide.

A “variant” as used herein, can alternatively or additionally be characterized by a certain degree of sequence identity to the parent polypeptide from which it is derived. More precisely, a variant in the context of the present invention exhibits “at least 80% sequence identity” to its parent polypeptide. Preferably, the sequence identity is over a continuous stretch of 20, 30, 40, 45, 50, 60, 70, 80, 90, 100 or more amino acids.

The expression “at least 80% sequence identity” is used throughout the specification with regard to polypeptide and polynucleotide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide. Preferably, the polypeptide in question and the reference polypeptide exhibit the indicated sequence identity over a continuous stretch of 20, 30, 40, 45, 50, 60, 70, 80, 90, 100 or more amino acids. Preferably, the polynucleotide in question and the reference polynucleotide exhibit the indicated sequence identity over a continuous stretch of 60, 90, 120, 135, 150, 180, 210, 240, 270, 300 or more nucleotides. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise.

The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/ or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/ or http://www.ebi.ac.uk/Tools/clustalw2/index.html. The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215: 403-410. BLAST polynucleotide searches are performed with the BLASTN program, score=100, word length=12, to obtain polynucleotide sequences that are homologous to those nucleic acids which encode the therapeutic proteins. BLAST protein searches are performed with the BLASTP program, score=50, word length=3, to obtain amino acid sequences homologous to the therapeutic proteins. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields. When percentages of sequence identity are referred to in the present application, these percentages are calculated in relation to the full length of the longer sequence, if not specifically indicated otherwise.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups:

(1) hydrophobic: Met, Ala, Val, Leu, Ile;

(2) neutral hydrophilic: Cys, Ser, Thr; Asn, Gln;

(3) acidic: Asp, Glu;

(4) basic: His, Lys, Arg;

(5) residues that influence chain orientation: Gly, Pro; and

(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe. Given the known genetic code, and recombinant and synthetic DNA techniques, the skilled scientist readily can construct DNAs encoding the conservative amino acid variants.

As used herein, “non-conservative substitutions” or “non-conservative amino acid exchanges” are defined as exchanges of an amino acid by another amino acid listed in a different group of the six standard amino acid groups (1) to (6) shown above.

A “biological activity” as used herein, refers to any activity a polypeptide may exhibit, including without limitation: enzymatic activity; binding activity to another compound (e.g. binding to another polypeptide, in particular binding to a receptor, or binding to a nucleic acid); inhibitory activity (e.g. enzyme inhibitory activity); activating activity (e.g. enzyme-activating activity); or toxic effects. It is not required that the variant or derivative exhibits such an activity to the same extent as the parent polypeptide. A variant is regarded as a variant within the context of the present application, if it exhibits the relevant activity to a degree of at least 10% of the activity of the parent polypeptide. Likewise, a derivative is regarded as a derivative within the context of the present application, if it exhibits the relevant biological activity to a degree of at least 10% of the activity of the parent polypeptide. The relevant “biological activity” in the context of the present invention is defined as an effect on living organism and processes examined.

As used herein, “operatively linked” means incorporated into a genetic construct so that expression control sequences effectively control expression of a coding sequence of interest.

As used herein, “genetically engineered” means that the host (or “carrier”) cell is transgenic for the polynucleotide or vector containing the polynucleotide.

As used herein, the term “vector” refers to a polynucleotide which is capable of being introduced or of introducing genes encoding proteins and nucleic acid comprised therein into a cell. In the context of the present invention it is preferred that the proteins encoded by the introduced polynucleotide are expressed within the cell upon introduction of the vector.

A polynucleotide encoding a “mature form” of a protein or polypeptide means that said protein or polypeptide contains all polypeptide elements that allow it to undergo some or all potential post- or cotranslational processes such as proteolytic processing, phosphorylation, lipidation and the like comprised in the state of the art such that said polypeptide or protein can correctly fold and carry out part or all of its wild-type function once it reaches its “mature form”.

The term “patient” means any subject in need of a treatment as described herein, and preferably encompass mammals and, more preferably equides (horses), and more preferably humans.

The term “wound” refers to any kind of damage of the tissue of the body, which is caused by events such as disease, injury, trauma or the like. The wound could be of various causes, such as but not limited to incisional, compression, acute, chronic, thermal, and infected.

The present invention will now be further described. Embodiments of the invention are defined in the independent claims. Preferred embodiments of the invention are defined in the dependent claims and the present specification.

In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Thus, in a first aspect the present invention is directed at an implantable device for releasing a combination of therapeutic proteins, wherein the device comprises cells preferably incorporated within the semipermeable container, simultaneously secreting a combination of at least two therapeutic proteins.

The present invention is directed at a device as defined above, wherein the amount and/or concentration of the therapeutic proteins secreted by the device is determined by the ratio of different therapeutic cells secreting different amounts of different therapeutic proteins.

Additionally or alternatively, the amount and/or concentration of the therapeutic proteins secreted by the device is/are determined by the expression and/or secretion of the different therapeutic proteins.

In a further preferred embodiment of the first aspect, the present invention is directed at a device as defined above, wherein each of the cells comprised in the device are prepared by the introduction of genes encoding therapeutic proteins under the control of constitutive or inducible promoter into a carrier cell.

In a further preferred embodiment of the first aspect, the present invention is directed at a device as defined above wherein said carrier cells are immortalized cell lines or cell lines capable of at least 60 doublings.

Preferably, the cells are eukaryotic cells, more preferably cells of mammal origin. More preferably, the cells belong to the same species as the patient to be treated.

In a further preferred embodiment of the first aspect of the present invention said carrier cells are selected from the group consisting of epithelial cells, mesenchymal stem cells, and retinal epithelial cells or cell lines.

In a further preferred embodiment of the first aspect of the present invention, the device defined above, said therapeutic proteins have an amino acid sequence which is identical to the ones existing in naturally occurring proteins or variants having an artificial amino acid sequence.

In a further preferred embodiment of the first aspect of the present invention is directed at said therapeutic proteins are preferably proteins of the following belonging to one of the protein families PDGF, VEGF, FGF, KGF, IGF, TGF, EGFL, and more preferably, said therapeutic proteins are selected from the group consisting of EGFL7, FGF-2, IGF-I, PDGF-B, TGF-β1 and VEGF-A or variants thereof.

In a further preferred embodiment of the first aspect of the present invention, said therapeutic proteins are secreted in a concentration range of 5 pg/ml to 20 ng/ml.

In a further preferred embodiment of the first aspect of the present invention, said container is a semipermeable hollow fiber containing said cells, allowing the exchange of nutrients and therapeutic proteins; or said container comprise semipermeable microcapsules containing said cells, allowing the exchange of nutrients and therapeutic proteins.

In a second aspect, the present invention is directed at the device as defined in the first aspect for use in the treatment of patients in need of being treated by the combination of therapeutic proteins defined above.

In a preferred embodiment of the second aspect of the present invention, the device as defined in the first aspect is used in the treatment of wounds, the promotion of angiogenesis, the promotion of bone remodeling, the treatment of peripheral artery disease, chronic artery disease, ischemia, organ repair after ischemic stroke, aortic/arterial wall injury, atherosclerosis, bone repair.

In a second aspect, the present invention is directed at the device as defined in the first aspect for use in the treatment of conditions in veterinary medicine: osteohondroses, equine anovulatory haemorrhagic follicles (AHFs), equine deep stromal abscesses, equine vasculature anomalies, tibial dyschondroplasia.

In a preferred embodiment of the second aspect of the present invention, the device as defined in the first aspect is used in a method for the treatment of wounds, the promotion of angiogenesis, the promotion of bone remodeling, the treatment of peripheral artery disease, chronic artery disease, ischemia, organ repair after ischemic stroke, aortic/arterial wall injury, atherosclerosis, bone repair.

In a preferred embodiment of the second aspect of the present invention, the present invention is directed at the device as defined in the first aspect for use in a method for the treatment of conditions in veterinary medicine: wounds, chronic artery disease, ischemia, organ repair after ischemic stroke, aortic/arterial wall injury, atherosclerosis, bone repair, osteohondroses, equine anovulatory haemorrhagic follicles (AHFs), equine deep stromal abscesses, equine vasculature anomalies, tibial dyschondroplasia.

In a third aspect, the present invention is directed at cells simultaneously secreting at least two therapeutic proteins for use in the manufacturing an implantable device for releasing a combination of therapeutic proteins as described in the first aspect.

The present invention describes a therapeutic device releasing (secreting) a combination of therapeutic proteins. The device contains cells which secrete therapeutic proteins, wherein the cells are preferably located in a container which enables the exchange of nutrients and therapeutic proteins. The cells are preferably genetically modified to express and secrete the therapeutic proteins in their environment.

Each therapeutic cell type preferably produces a certain amount of one or more specific therapeutic proteins and is preferably prepared by the introduction of genes encoding therapeutic proteins under the control of constitutive or inducible promoter into a carrier cell. Where possible, clones of therapeutic cells are selected to insure proper and relatively stable secretion of therapeutic proteins. Preferably, these clones are banked. Preferably, each therapeutic cell type was prepared to secrete one therapeutic protein in a defined manner, however the combination and concentration of the secreted therapeutic proteins is then preferably controlled by the number and the ratio of different therapeutic cell types incorporated in the semi-permeable container. The combination and concentration of the therapeutic protein is defined by controlling the expression and/or transport of the therapeutic proteins in the cells in the semi-permeable container. The definition of the combination and concentration of the therapeutic proteins by the ratio of different therapeutic cell types in a cell mixture incorporated in the semi-permeable container is most preferred.

To illustrate the approach, a hypothetical case is described. From state-of-the-art it is known that therapeutic proteins A, B, C, D and E have positive effects in treatment of specific condition termed X. Plasmids carrying ORFs A or B or C or D or E and antibiotic resistance genes were introduced into a carrier cell line via transfection. Stable integration of DNA encoding A or B or C or D or E is achieved by growing transfected cells in cell culture medium supplemented by appropriate antibiotic. Several clones for each therapeutic protein are selected. Concentration of therapeutic proteins is followed over appropriate period of time to determine the characteristics of a particular therapeutic cell clone (regarding amount of secreted protein and stability). Therapeutic cell clones are banked until further use.

In the meanwhile several combinations of therapeutic proteins (of various compositions regarding the types of proteins and their concentration) are tested in in vitro and ex vivo models of condition X. We determined that optimal combination or combination range of therapeutic proteins is A (a pg/ml), B (b pg/ml), C (c ng/ml) and E (e pg/ml). Further different therapeutic cell types, which each secrete a defined amount of specific therapeutic protein are mixed in appropriate ratio (nₐ of cell type secreting protein A, n_{b} of cell type secreting protein B, n_{c} of cell type secreting protein C and nₑ of cell type secreting protein E) to achieve previously defined optimal combination of therapeutic proteins or several well performing combinations. These cells are introduced into a container and implanted into a patient. The healing process is further followed on appropriate time-scale.

The main advantage of the present invention is that it is possible to easily modify the combination of therapeutic proteins to suit for the treatment of each separate condition or even patient while the costs of production are not significantly increased.

The implantation of the device of the invention enables local (in situ), continuous and simultaneous delivery of physiological amounts of therapeutic proteins at site where needed most, while avoiding frequent and systemic applications of therapeutic proteins which often led to serious adverse effects. Additionally, several therapeutic proteins in low amounts are used to achieve the maximal healing effect, which also lowers the possibility of adverse side-effects caused by application of non-physiological amounts of single therapeutic protein.

Another advantage is that the production of therapeutic proteins by the used cells is high, thus lower numbers of cells secreting therapeutic proteins are needed to achieve physiological concentrations of therapeutic proteins, consequently leading to lower amounts of other (non-monitored) proteins secreted.

The cell lines to be used in the present invention (“carrier cell lines”) are preferably immortal or immortalized cell lines, which can be easily genetically manipulated and banked, which is much cheaper in contrast to the isolation and manipulation of stem cells. Additional advantage of our system over stem cells is that the composition of therapeutic protein mixture is defined and controlled, while for stem cells it is not possible to define how they react in vivo.

### Therapeutic Proteins

Therapeutic proteins in the sense of the present invention are either proteins, which exist in nature, such as unmodified growth factors, or are designed therapeutic proteins, such as single-chain variable fragments of naturally occurring proteins or a variants thereof. Therapeutic proteins are preferably introduced into a carrier cell line via genetic manipulation techniques well known to the experts in the field.

Therapeutic proteins exert their biological activity via different healing mechanisms. For example, the epidermal growth factor is known to stimulate keratinocyte and fibroblast proliferation, transforming growth factor alpha (TGF-α) is chemotactic for keratinocytes and fibroblasts, TGF-β1 and TGF-β2 promote angiogenesis and up-regulate the production and inhibit degradation of collagen, while their antagonist TGF-β3 promotes scarless wound healing. TGF-β1 suppresses immune system. The therapeutic proteins of the vascular endothelial growth factor (VEGF) family promote angiogenesis, fibroblast growth factors (FGFs) promote angiogenesis, granulation, and epithelialization. The platelet-derived growth factor (PDGF) is chemotactic for granulocytes, macrophages and fibroblasts. The keratinocyte growth factor (KGF) stimulates keratinocyte migration, proliferation and differentiation. The hepatocyte growth factor (HGF) promotes progenitor cell mobilization, induces angiogenesis and cell proliferation. The insulin-like growth factor (IGF) family induces cell proliferation and inhibits apoptosis, monocyte chemotactic protein-1 induces angiogenesis, and inhibits apoptosis. The brain-derived neurotrophic factor (BDNF), ciliary neurotrophic factor (CNTF), glial cell-derived neurotrophic factor (GDNF) and the nerve growth factor (NGF) promotes neuronal cell survival. The biological activity of the therapeutic proteins as well as functional variants thereof can be measured by assays well-known to the person skilled in the art, such as various migration and proliferation assays in vitro (as described by Schreier et al., 1993), measuring of binding protein activity, their effect on DNA synthesis, protein accumulation and mouse models of particular condition.

Therapeutic proteins are not only growth factors, but also other proteins with biological activity, such as but not limited to protease inhibitors or immune receptor antagonists (e.g. anakinra).

Therapeutic proteins used in the present innovation can be of form, in amino acid sequence and protein secondary and tertiary structure identical to naturally present, or may be modified or designed for improved action. For example, chimeric proteins can be formed by fusion of different therapeutic proteins. For example, the preparation of a fusion of receptor-binding parts of vascular endothelial growth factor—angiopoietin with improved properties for angiogenesis is described by Anisimov (Anisimov et al., 2013). Another example is given by Martino et al., who produced growth factors with enhanced affinity for extracellular matrix proteins (Martino et al., 2014).

Therapeutic proteins are also bioactive molecules, not present in nature, such as single chain variable fragments, recombinant antibodies, peptides acting as antagonists of unwished cellular processes, such as TNF-α a neutralizing antibodies or soluble receptors for IL-1b.

Preferably, the therapeutic proteins are selected from combinations of two or more proteins belonging to the protein families PDGF, VEGF, FGF, KGF, IGF, TGF, EGFL and variants of proteins belonging to these protein families. More preferably, the therapeutic proteins are selected from EGFL7, FGF-2, IGF-I, PDGF-B, TGF-β1 and VEGF-A or variants thereof. In a further preferred embodiment of the invention the secreted therapeutic proteins are a combination of EGFL7, FGF-2, IGF-I, PDGF-B, TGF-β1 and VEGF-A corresponding to SEQ ID Nos 1 (UniProt acc.no.: Q9UHF1), 2 (UniProt acc.no.: P09038), 3 (UniProt acc.no.: P05019), 4 (UniProt acc.no.: P01127), 5 UniProt acc.no.: P01137), 6 (UniProt acc.no.: P15692) or variants thereof.

Concentrations of therapeutic proteins released from said cell device are in physiological range for natural proteins. Preferred effective concentration ranges for particular therapeutic proteins are listed in table 1. For therapeutic proteins, not listed in the table, effective concentration range is between low (e.g. 5) pg/ml range to 100 μg/ml range, more preferably from low pg/ml to several ng/ml range. To achieve said effective concentrations of released therapeutic proteins, a total of 1*10³-5*10³ cells will be packed per mm³ of cellular device. The techniques for implementation of the said device are well known and routine to skilled artisans.

TABLE 1
Effective concentration in the vicinity of implanted cellular device

| | Low-high concentration |
|---|---|
| Therapeutic protein | range |
| VEGF | 50 pg/ml-10 ng/ml |
| PDGF | 5 pg/ml-1 ng/ml |
| TGFb1 | 10 pg/ml-0.5 ng/ml |
| FGF | 5 pg/ml-2 ng/ml |
| EGFL | 25 pg/ml-10 ng/ml |
| IGF | 20 pg/ml-5 ng/ml |

### Carrier Cell Lines

The present invention relates to cells releasing therapeutic proteins. Preferably, the invention is directed at cell lines, which are genetically modified to release one or more therapeutic protein.

A carrier cell line is a parent cell line, which is preferably genetically modified in the present invention to produce and release one or more therapeutic protein. Preferably, carrier cell lines are of human origin for applications in medicine and should secrete low amounts of non-desired proteins, since even if immunologically isolated in a container, non-autologous cells secrete antigens, which trigger immune rejection of the graft (de Groot et al., 2004). For applications in veterinary medicine the carrier cell lines may preferably be of other corresponding species origin.

Preferably, carrier cell lines to be used in the present invention could be spontaneously immortal or immortalized via insertion of a heterologous immortalization gene, yet must not be tumorigenic. Such cell line is for example but not limited to human embryonic kidney cell lines. Another possibility to further decrease safety risks is to incorporate safety kill-switches, such as thymidine kinase introduction into parent carrier cell line. Preferably, an operon encoding thymidine kinase of herpes simplex virus type 1 (HSV-TK) could be stably introduced into our carrier line. This protein is not secreted and remains within the cells. The resulting cell line will stably express thymidine kinase, which produces highly cytotoxic compound in the presence of a ganciclovir or its analogs. More preferably, mutants of D. melanogaster thymidine kinase with decreased LD₅₀ to several nucleoside analogues (WO 01/88106) could be used.

Non-immortalized cells could also be used, However, the applicability of the cell line in the present invention depends on its proliferation capacity. Preferably, the cell line should be capable of a minimum of 60 divisions, more preferably 90 divisions or more. Preferably, carrier cell lines should have a life span which is long enough for their therapeutic use according to the present invention.

The carrier cell line is preferably a contact-inhibited cell line which stops its proliferation once certain cell density inside a container is reached. Another possibility, in cases where carrier cell line lost contact inhibition, is to irradiate cells prior placement into a container. Irradiated cells stop their proliferation; but they are still able to produce and secrete therapeutic proteins.

Possible carrier cells could be mesenchymal stem cells, since they are hypoimmunogenic and have immunomodulatory properties, they could in principle be immortalized and engineered to secrete the desired protein.

Possible carrier cell lines could be epithelial cells, which are contact inhibited. It is possible to isolate primary retinal pigment epithelial cells from mammalian retina known to the skilled artisans.

For applications were long term treatment is needed carrier cell line should also be hardy to survive harsh conditions such as avascular tissue or inflammed tissue.

U.S. Pat. No. 6,361,771 describes testing of a variety of mammalian cell lines for compatibility with long-term implantation criteria and finds the ARPE-19 cell line (ATCC no. CRL-2302) as best performing. Moreover, this cell line has already been used as a carrier cell line in clinical studies to deliver NGF (US 2008/0286323). Other possible carrier cells include human immortalized endothelial or fibroblast or astrocyte cell lines.

### Construction of Therapeutic Cells.

Methods for preparation of gene constructs and introduction into mammalian cell lines are well known to experts in the field. Well established cloning techniques can be used to prepare constructs in pcDNA family vectors, pFLAG family vector, pCL family vector, lentiviral expression vector family, retroviral expression vectors such as pMXs family vectors, BAC and other mammalian vectors with CMV, SV40, EF1a and CAG promoters. Therapeutic protein ORFs obtained from commercial suppliers (e.g. Origene, Sino biological), custom synthesized or isolated from various organisms, can be cloned under CMV SV40, EF1a and CAG or other mammalian promoters. Usage of strong promoters and other approaches known to the skilled in the art provide the production of therapeutic protein in high concentrations. Cells could be transfected using transfection reagents such as PEI, lipofectamine 2000, Fugene, or the gene will be introduced via electroporation or viral transduction. 24-48 h after gene introduction, cells will be either treated with antibiotic to select for clones stably expressing therapeutic proteins. Several clones of therapeutic cells for each therapeutic protein will be selected for further work.

### Containers

The cellular device of this invention contains cells producing therapeutic proteins in one or more semi-permeable containers. The function of the semi-permeable container(s) is to disable uncontrollable cell spread upon implantation and to prevent or minimize host immune response and implant rejection. The container is/are made from material which is compatible with host and does not trigger host response. The container is/are made from material, which allows for diffusion of therapeutic proteins, exchange of nutrients and metabolites, but does not allow the entrance of host immune molecules into the container. Semipermeable container(s) could be made of polyacrylates, polyurethanes, polystyrenes polyvinylidenes, polyamides, polyvinyl chloride copolymers, cellulose acetates, cellulose nitrates, polysulfones, polyphosphazenes, polyacrylonitriles, as well as other materials known to the experts in the field. Preferably, the container(s) is/are made of semipermeable hollow fiber such as described in U.S. Pat. No. 5,284,761. Preferably, the container(s) is/are made of modified polyvinylidene difluoride hollow fiber (mPVDF, Spectrum laboratories). This type of container has been already validated in vivo and for the pharmaceutical drug screening (Hollingshead et al., 1995). The container(s) may also be microcapsules made of alginate in combination with polylysine or chitosan, especially for implantation into positions, where space is very limited. The container(s) may also be filled by a scaffold or a matrix to support cell survival.

### Preparation of Cellular Devices for Local (In Situ) Therapy.

The device of the present invention comprise cells producing therapeutic proteins, which are located in a semi-permeable container. Each therapeutic cell line produces at least one specific therapeutic protein and more than one therapeutic cell type is preferably combined (i.e. mixed) in the device with at least one other, preferably three, more preferably four, more preferably five, and even more preferably 6 and more cell types producing therapeutic proteins. The combination of cell types and of therapeutic proteins is determined for each therapeutic application. The ratio of therapeutic proteins is further adjusted.

Alternatively, cell lines may be used which express two or more therapeutic proteins and wherein the ratio of the therapeutic proteins may be controlled by regulation of the expression, maturation and/or secretion of therapeutic protein.

One of the major advantages of this invention over existing state-of-the-art platforms is that its usage is not limited to a single medical condition, but could in principle by the selection of appropriate container and appropriate combination of cell types secreting therapeutic proteins be used for any condition, where local administration of therapeutic proteins is effective. The concentrations of therapeutic proteins are at physiological levels thus no or less adverse effects are expected. Further, in a preferable embodiment, cell lines secreting soluble proteins are bankable and immortal cell lines, which allows for large scale cultivation decreasing the cost.

Herein described device is used for treatment of disorders, defects, diseases, injuries and other conditions where therapeutic proteins are effective. The device is used for treatment of for example but not limited to chronic wounds, burns, peripheral artery disease, chronic artery disease, ischemia, organ repair after ischemic stroke, aortic/arterial wall injury, atherosclerosis, bone repair. The device is used for treatment of conditions in veterinary medicine, such as osteohondroses, equine anovulatory haemorrhagic follicles (AHFs), equine deep stromal abscesses, equine vasculature anomalies, tibial dyschondroplasia.

### Wound Healing.

Normal wound healing involves a series of coordinated events, occurring in three phases: inflammatory (0-3 days), proliferative (3 days-2 weeks) and remodeling (up to 2 years). Inflammatory phase occurs immediately after injury, coagulation cascade is activated to minimize blood loss. Neutrophils and later also monocytes/macrophages are recruited to the wound. These cells remove foreign material and fight bacteria. They are recruited via chemokines. Macrophages release numerous potent growth factors such as TGF-β, FGF and EGF, which further activate keratinocytes, fibroblasts and endothelial cells. The proliferative phase is characterized by fibroblast recruitment, extracellular matrix deposition and formation of granulation tissue. Factors particularly important in this phase are TGF-β, which stimulates production of matrix components by fibroblasts and of the tissue inhibitor of metalloproteinases. Other important trophic factors include FGFs, interleukins and TNF-α. TGF-β and EGF stimulate epithelialization. VEGF, basic FGF and TGF-β stimulate angiogenesis. Remodeling phase is characterized by reorganization of the matrix, the underlying connective tissue shrinks in size and brings the wound margins closer together, which is regulated by PDGF, TGF-β and FGFs. When the wound is healed, macrophages and fibroblasts undergo apoptosis, angiogenesis stops and blood flow to the area declines.

In chronic wounds, the process does not go through the three normal stages. Infection, neuropathy and impaired vascular supply contribute to the formation of the diabetic wound. Migration and proliferation of specific cell types are altered thus also growth factor production is impaired. Chronic inflammation leads to persistent increase in pro-inflammatory cytokines by various immune and non-immune cells, which is proposed to block normal cytokine response. Impaired angiogenic response halts granulation tissue formation and regeneration and leads to prolonged hypoxia. Acute hypoxia in normal wound leads to activation of hypoxia inducible factor-1 (HIF-1α), hyperglycemia in diabetic patients however affects the stability and activation of HIF-1α, which is further reflected in suppression of PDGF, VEGF and TGF-β.

The device described in this invention enables accelerated wound healing and reduces the incidence of wound failure by sustained release of physiological concentrations of a combination of therapeutic proteins for a desired period of time. The device for wound healing preferably comprises a combination of cells secreting therapeutic proteins enclosed into semi-permeable container. Preferably, the cellular device is implanted close to the site of wound. The wound could be of various causes, such as but not limited to incisional, compression, acute, chronic, thermal, and infected. The container could be of various sources compatible with the criteria described above. In one specific embodiment, the container is made of mPVDF hollow fiber, cell lines stably expressing a combination of therapeutic proteins are placed into the container, which is afterwards thermally sealed. The device is than placed at the preferred site, for example in close proximity of the wound or directly into the wound.

A combination (i.e. a mixture) of cells secreting effective amounts of therapeutic proteins will be determined by the patient's attending physician or veterinarian and will depend on the specificity of the wound type and size, physical condition of the patient and other important characteristics. The said cell lines each stably express one therapeutic protein such as but not limited to trophic factors EGF, TGFs, VEGFs and PDGFs, KGF. The effective amounts of therapeutic proteins are achieved by combining the cells secreting various therapeutic proteins in appropriate cell number and/or expression ratios.

### Angiogenesis.

The treatment of ischemic cardiovascular and cerebrovascular disorders and the like should aim at the restoration of functional blood flow to ischemic tissue and organs. Recovery depends on establishing collateral networks that sufficiently supply oxygenated blood to specialized cells. The organism has developed some compensatory mechanisms, in which low levels of oxygen can be improved. This occurs due to the mechanism of vasodilatation, angiogenesis, arteriogenesis, vascular remodeling and increased hematopoiesis. Angiogenesis is the process of sprouting newly formed capillaries from existing blood vessels. The key cells for this sprouting angiogenesis are the endothelial cells. They are primarily responsible for capillary growth, migration and organization of vessel lumen. The helper cells for stability maintenance of newly formed blood vessels in capillary are pericytes and smooth muscle cells in arterioles, venules, artery and veins. The other form of angiogenesis is an intussusceptive angiogenesis, where two blood vessels form by dividing one existing capillary. Sprouting angiogenesis, in which new blood vessels are formed, is continued from vasculogenesis, a process, where endothelial cells precursors originate from mesoderm and form tubes into primary vascular plexus. So the first blood vessels in embryo form through vasculogenesis, after which angiogenesis and arteriogenesis are responsible for most blood vessels formation. Angiogenesis process starts due to the injury of the tissue (mechanical stimulation) or due to the chemical stimulation via growth factors. Key signaling molecules to vascular morphogenesis and therefore to angiogenesis are VEGF, notch, angiopoetins, ephrins, TGF-β and PDGF. Normally, blood vessels are covered with basement membrane, consisting primarily of laminins, collagen type IV, nidogens, and the heparan sulfate proteoglycan. In early stages of angiogenesis, basal membrane is degraded due to the angiogenic cytokine VEGF signaling, induced by wounding and ischemia state. Following the membrane degradation is vascular sprouting, a process in which endothelial cells are invading vessel wall and extracelluar matrix (ECM) is formed due to the hyperpermeability of blood vessels. Within the vascular sprout there is lumen formation, thereby creating vascular tubes and covering it with basal membrane and pericytes, that finally results in newly formed capillary. Key molecules in angiogenesis are angiogenic growth factors. All large clinical trials that are focused into treating ischemic conditions are designed basically as monotherapy, but this is not sufficient for constructing functional arterial network. It is known that newly formed blood vessels have to stabilize and mature. This process depends on costimulatory activity of angiogenic growth factors, combination of PDGF-B and VEGF showed improved therapeutic benefits due to proper maturation of blood vessels, but in cases, where only VEGF was used as a treatment choice, the newly formed blood vessels regressed.

The device described in this invention enables accelerated angiogenesis in a safe physiological manner. The device induced the formation of new blood vessel collateral networks and therefore supply ischemic tissue and organs with oxygenated blood and reduce the hypoperfusion and reperfusion injuries.

### EXAMPLES

### Example 1

### Preparation of DNA Constructs

DNA sequences for therapeutic proteins described above were either ordered from supplier, e.g. “Sino biological Inc.”, or designed from amino-acid sequences of the selected protein domains using tool Designer from DNA2.0 Inc. that enables the user to design DNA fragments and optimize expression for the desired hosts (e.g. human cells) using codon optimization. DNA encoding the genes were then ordered from GeneArt or GeneScript, cut out with restriction endonucleases (restriction enzymes) and cloned into the appropriate vector containing necessary regulatory sequences known to the experts in the field. Vectors used include commercial vectors of pcDNA, pMXs etc. carrying all necessary features such as antibiotic resistance, origin of replication and multiple cloning site.

Molecular biology methods (DNA fragmentation with restriction endonucleases, DNA amplification using polymerase chain reaction-PCR, PCR ligation, DNA concentration detection, agarose gel electrophoresis, purification of DNA fragments from agarose gels, ligation of DNA fragments into a vector, transformation of chemically competent cells E. coli, isolation of plasmid DNA with commercially available kits, screening and selection) were used for preparation of DNA constructs. All procedures were performed under sterile conditions (aseptic technique). DNA segments were characterized by restriction analysis and sequencing. Molecular cloning procedures are well known to the experts in the field and are described in details in molecular biology handbook (Sambrook J., Fritsch E. F., Maniatis T. 1989. Molecular cloning: A laboratory manual. 2nd ed. New York, Cold Spring Harbor Laboratory Press: 1659 p.).

### Example 2

### Preparation of Transient and Stable Cell Lines

Selected carrier cell lines HEK293 (ATCC CRL-1573), NIH 3T3 (ATCC CRL-1573) and ARPE-19 were transfected with prepared constructs for the constitutive production of therapeutic proteins via lipofectamine 2000 and polyethylene imine reagents. 24 h to 36 h post transfection the production of therapeutic proteins was assessed in cell culture supernatant by commercially available ELISA tests.

Therapeutic cell lines were generated by the addition of selective marker (antibiotic such as neomycin, puromycin, zeocin or blasticidine). Several clones exhibiting high therapeutic protein secretion level, growth characteristics and stability were selected for each therapeutic protein. Stocks of therapeutic cell lines were frozen and stored in liquid nitrogen vapor phase.

Mouse fibroblasts NIH 3T3 were plated at low density in 10 cm petri dish and transfected with plasmids encoding soluble factors (EGFL7, FGF-2, IGF-I, PDGF-B, TGF-β1 and VEGF-A). One day after transfection, cells were trypsinized and antibiotic Geneticin (G418) in concentration 1.2 g/l was added to the cells. Growth medium containing G418 was changed three times a week until the selection was complete. Several clones for each factor were selected for further work.

The secretion of FGF-2, IGF-1 and PDGF-B from stable NIH 3T3 cell lines was monitored by commercially available ELISA assays. Stable cell lines were plated at low density in 6-well plates. Concentration of growth factors was measured on day 1 and day 14. Concentrations on day 1 were 7, 4 and 12 ng/ml for FGF-2, IGF-1 and PDGF-B. Concentrations on day 14 were 56, 28 and 210 ng/ml for FGF-2, IGF-1 and PDGF-B, respectively. When adjusted for the number of cells, we can conclude that the secretion rate of therapeutic proteins is quite stable. Additionally, we estimate that the secreted amount rate of these therapeutic proteins is at least 10²-10⁵-times larger than secretion by stem cells.

### Example 3

### Gap Migration Assay of Therapeutic Protein Combinations

The effect of each therapeutic protein and their combinations was first tested in gap migration assay on established cell line NIH 3T3.

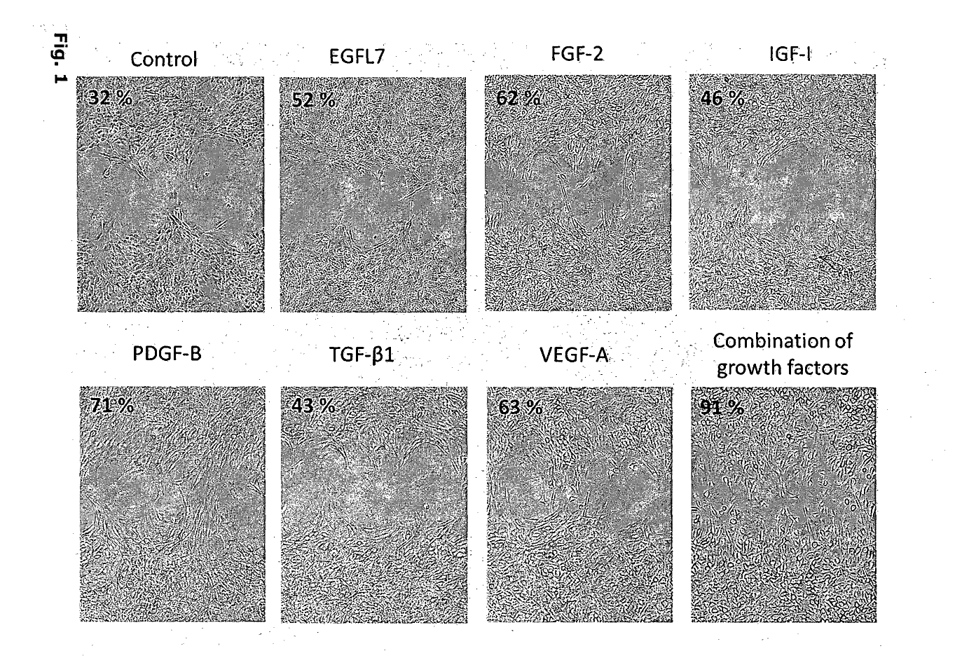

One day before the wound scratch assay, NIH 3T3 cells (5*10⁴ cells/well) were seeded onto 8 well μ-slide with inserted culture insert (Ibidi). Day after seeding a confluent monolayer of cells was formed. Insert was removed leaving a 500 μm gap. Supernatants containing a single therapeutic protein or a combination in concentrations ranging from 1 pg/ml to several ng/ml were added. Gap closure was measured after 6, 12 and 24 h. As seen in FIG. 1, the addition of therapeutic proteins had a positive effect on wound closure compared to control without growth factors. Addition of a combination of 6 growth factors (EGFL7, FGF-2, IGF-I, PDGF-B, TGF-β1 and VEGF-A) had the best effect on gap closure.

### Example 4

### Testing of Therapeutic Protein Combinations in Skin Organ Slices

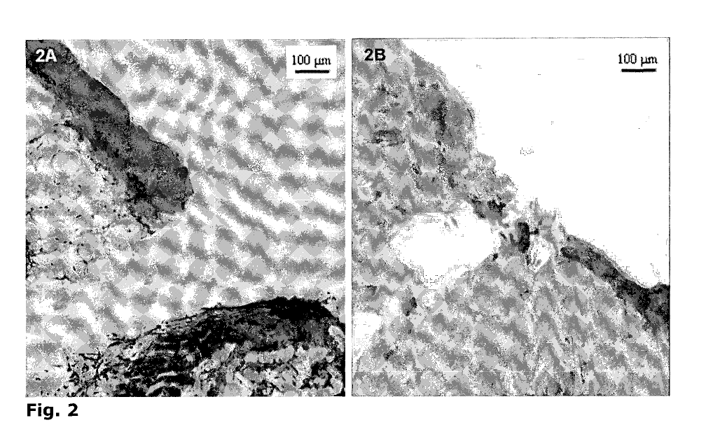

Selected combinations of therapeutic proteins were tested in skin organ punches. Full thickness circular excisions from healthy non-wounded skin were obtained from C57BL/6 OlaHsd mice. Using punch biopsy scalpel (8 mm) skin was excised into round pieces and wounded with scalpel. Samples were cultivated using 12 well plate filled with DMEM containing 10% FBS and 1% penicillin/streptomycin. Skin was only partial submerged in medium allowing the keratinocyte layer to be in direct contact with air. Medium was changed every second day and mixture of soluble factors (5 μl) was added to the wounded area daily. Skin was cultured for 7 days and then fixed using Histofix (Roth) preservative for histology assessment. A standard HE-staining was performed. We observed that 6 soluble factors (EGFL7, FGF-2, IGF-I, PDGF-B, TGF-β1 and VEGF-A) enhance wound healing in skin punches (see FIG. 2).

### Example 5

### Construction and Experimental Validation of Therapeutic Device for Wound Healing

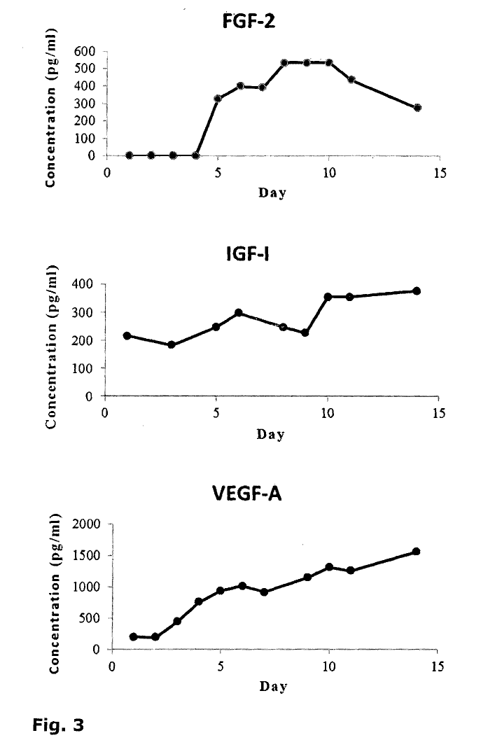

A total of 3*10⁴ of cells secreting EGFL-7, FGF-2, IGF-I, PDGF-B, TGF-β1, and VEGF-A were inserted into mPVDF hollow fiber (Spectrum labs), the fiber was thermally sealed and the device was placed into culture dish with DMEM growth medium. Containers were inspected daily to determine if cells are present outside the hollow fiber. Amounts of soluble factors were followed daily for 14 days, cell culture medium was also tested for the release of lactate dehydrogenase, a marker of necrosis. During the time of observation (14 days) no cells were observed outside the container. From 0-4 days concentrations of factors increased, when they reached the plateau, which was stable until the end of experiment (14^{th} day) (FIG. 3). We observed no increase in the lactate dehydrogenase activity in the supernatant during this time interval. On day 14 the viability of the cells extracted from the container was 91% determined by trypan blue exclusion test.

### Example 6

### Analysis of the Device Performance in Animal Wound Healing Assay in a Mouse Model

The wound healing experiments were performed according to regulations of Administration of the Republic Slovenia for Food Safety, Veterinary and Plant Protection (Ministry of Agriculture and the Environment) and National Ethical Committee for laboratory animal research. Mice C57BL/6 OlaHsd were premedicated with a xylazine/ketamine mixture. The anesthesia was maintained with inhalation of 1.5% isoflurane. Wound splint was stitched to the back of the mice and wound was made using punch biopsy scalpel (8 mm). Hollow fiber containing combination of 6 cell lines, producing therapeutic proteins (EGFL-7, FGF-2, IGF-I, PDGF-B, TGF-β1, and VEGF-A) was inserted into the wound and whole wound area was covered with transparent wound patch. After 7 days wound healing was strongly improved compared with a non-treated control group. The wound closure due to the formation of neoepithelium is greater in animal, treated with cellular device then in animals, treated with carrier cells or control. The wound area in animal with cellular device was smaller than in non-treated and in carrier cell line treated animal due to the formation of newly connective tissue (larger amounts of fibrin production and accumulation and therefore better wound healing). The HE staining confirmed the clinical results in animals. The skin samples from C showed completely closed wound due to the fibrin and reticulus fiber accumulation and cell infiltration (macrophages and fibroblasts). In the samples from A in B there is lesser wound closure, that results from greater epithelial gap between wound margins.

### Example 7

### Analysis of the Device Performance on Postnatal Arteriogenesis and Angiogenesis

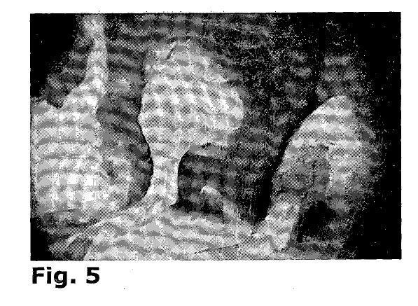

The effect of cellular device on postnatal arteriogenesis and angiogenesis was established using a mouse model of unilateral hind-limb ischemia, which is based on ligation and excising the femoral artery. Mice C57BL/6, aged 10-12 weeks were used. Mice were anesthetized with ketamine-ksilazine mixture and the hind-limb was surgically prepared. The surgical incision was made from the knee towards the medial thigh. Using retractor and forceps the deep femoral bundle was located. Then the femoral artery, vein and nerve were identified and gently separated from each other. Using 7/0 nonabsorbable surgical suture material the femoral artery was ligated immediately distal to the origin of the deep femoral branch, right after the branching of epigastric artery and profound femoral artery. An additional surgical knot was placed under the ligation knot. The femoral artery was then double ligated near the popliteal region. The ligated portion of femoral artery was then excised. Near the ischemic region cellular device was implemented. In the control group of mice there was just unilateral hind-limb ischemia performed with no cellular device implementation. The skin was sutured using 5/0 nonabsorbable surgical suture material. Mice were monitored daily. After the 7 days, mice were humanely euthanized and the gastrocnemius muscle was harvested. The mouse with the implanted cellular device showed better clinical appearance in contrast to the mouse with no cellular device. The cellular device improved functionality of the hind-limb after ischemic surgical procedure. The hind-limb from the mouse with implanted cellular device showed less swelling and sub dermal edema then mouse with no cellular device (FIG. 5). The tissue was preserved and then hematoxylin-eosin staining was performed, determining the occurrence of necrosis in mice without cellular device and determining the newly formed blood vessels in animals with cellular device. HE staining in gastrocnemius muscle from the mouse with implanted cellular device showed less inflammation resulting from reduced number of infiltrating immune cells (neutrophil granulocyte, lymphocyte), lesser accumulation of fibrin fibrils and necrotic lesions comparing to the gastrocnemius muscle from mouse with no cellular device that showed greater inflammation and necrotic lesions. In the sample from the mouse with cellular device there was greater formation of newly blood vessels then in control sample. There was greater number of small new forming arteries. The immunostaining with anti-CD31 (Platelet endothelial cell adhesion molecule; PECAM-1), determining the angiogenesis, was also conducted. The immunohistochemistry sample of the gastrocnemius muscle from the mice with implanted cellular device showed greater expression of CD31 then gastrocnemius muscle from the control mouse, that had unilateral hind-limb ischemia with no implanted cellular device.

### CITATION LIST

WO 2011/123779
US 2011/0020291
U.S. Pat. No. 5,487,889
WO 01/88106
U.S. Pat. No. 6,361,771
US 2008/0286323
U.S. Pat. No. 5,284,761
Anisimov, A., Tvorogov, D., Alitalo, A., Leppanen, V. M., An, Y., Han, E. C., Orsenigo, F., Gaal, E. I., Holopainen, T., Koh, Y. J., Tammela, T., Korpisalo, P., Keskitalo, S., Jeltsch, M., Yla-Herttuala, S., Dejana, E., Koh, G. Y., Choi, C., Saharinen, P. and Alitalo, K. (2013) Vascular endothelial growth factor-angiopoietin chimera with improved properties for therapeutic angiogenesis. Circulation, 127, 424-434.
Barcelos, L. S., Duplaa, C., Krankel, N., Graiani, G., Invernici, G., Katare, R., Siragusa, M., Meloni, M., Campesi, I., Monica, M., Simm, A., Campagnolo, P., Mangialardi, G., Stevanato, L., Alessandri, G., Emanueli, C. and Madeddu, P. (2009) Human CD133+ progenitor cells promote the healing of diabetic ischemic ulcers by paracrine stimulation of angiogenesis and activation of Wnt signaling. Circ Res, 104, 1095-1102.
Barinaga, M. (1994) Neurotrophic factors enter the clinic. Science, 264, 772-774.
de Groot, M., Schuurs, T. A. and van Schilfgaarde, R. (2004) Causes of limited survival of microencapsulated pancreatic islet grafts. J Surg Res, 121, 141-150.
Di Santo, S., Yang, Z., Wyler von Ballmoos, M., Voelzmann, J., Diehm, N., Baumgartner, I. and Kalka, C. (2009) Novel cell-free strategy for therapeutic angiogenesis: in vitro generated conditioned medium can replace progenitor cell transplantation. PLoS One, 4, e5643.
Emerich, D. F., Orive, G., Thanos, C., Tornoe, J. and Wahlberg, L. U. (2013) Encapsulated cell therapy for neurodegenerative diseases: From promise to product. Adv Drug Deliv Rev., 67-68, 131-141.
Hollingshead, M. G., Alley, M. C., Camalier, R. F., Abbott, B. J., Mayo, J. G., Malspeis, L. and Greyer, M. R. (1995) In vivo cultivation of tumor cells in hollow fibers. Life Sci, 57, 131-141.
Javazon, E. H., Keswani, S. G., Badillo, A. T., Crombleholme, T. M., Zoltick, P. W., Radu, A. P., Kozin, E. D., Beggs, K., Malik, A. A. and Flake, A. W. (2007) Enhanced epithelial gap closure and increased angiogenesis in wounds of diabetic mice treated with adult murine bone marrow stromal progenitor cells. Wound Repair Regen, 15, 350-359.
Krieglstein, K., Henheik, P., Farkas, L., Jaszai, J., Gaiter, D., Krohn, K. and Unsicker, K. (1998) Glial cell line-derived neurotrophic factor requires transforming growth factor-beta for exerting its full neurotrophic potential on peripheral and CNS neurons. J Neurosci, 18, 9822-9834.
Lee, Y. S., Danandeh, A., Baratta, J., Lin, C. Y., Yu, J. and Robertson, R. T. (2013) Neurotrophic factors rescue basal forebrain cholinergic neurons and improve performance on a spatial learning test. Exp Neurol, 249, 178-186.
Lynch, S. E., Nixon, J. C., Colvin, R. B. and Antoniades, H. N. (1987) Role of platelet-derived growth factor in wound healing: synergistic effects with other growth factors. Proc Natl Acad Sci USA, 84, 7696-7700.
Martino, M. M., Briquez, P. S., Guc, E., Tortelli, F., Kilarski, W. W., Metzger, S., Rice, J. J., Kuhn, G. A., Muller, R., Swartz, M. A. and Hubbell, J. A. (2014) Growth factors engineered for super-affinity to the extracellular matrix enhance tissue healing. Science, 343, 885-888.
Schreier, T, Degen, E, Baschong, W. (1993) Fibroblast migration and proliferation during in vitro wound healing. A quantitative comparison between various growth factors and a low molecular weight blood dialysate used in the clinic to normalize impaired wound healing. Res Exp Med (Berl)., 193(4), 195-205.
Wu, Y., Chen, L., Scott, P. G. and Tredget, E. E. (2007) Mesenchymal stem cells enhance wound healing through differentiation and angiogenesis. Stem Cells, 25, 2648-2659.
Zanin, M. P., Pettingill, L. N., Harvey, A. R., Emerich, D. F., Thanos, C. G. and Shepherd, R. K. (2012) The development of encapsulated cell technologies as therapies for neurological and sensory diseases. 3 Control Release, 160, 3-13.
Zhang, Y., Ingram, D. A., Murphy, M. P., Saadatzadeh, M. R., Mead, L. E., Prater, D. N. and Rehman, J. (2009) Release of proinflammatory mediators and expression of proinflammatory adhesion molecules by endothelial progenitor cells. Am J Physiol Heart Circ Physiol, 296, H1675-1682.

1. An implantable device for releasing therapeutic proteins, wherein the device comprises cells enclosed in a container, which simultaneously release at least two therapeutic proteins, wherein the amount and the ratio of the therapeutic proteins released by the device are determined by the number and the ratio of different cells in the container which differ in that they secrete different therapeutic proteins.

2. The device according to claim 1, wherein each of the cells comprised in the device are prepared by the introduction of at least one gene encoding a therapeutic protein under the control of a constitutive or inducible promoter into a carrier cell.

3. The device according to claim 2, wherein said carrier cells are immortalized cell lines or cell lines capable of at least 60 doublings.

4. The device according to claim 2, wherein said carrier cells are selected from the group consisting of epithelial cells, mesenchymal stem cells and retinal epithelial cells or cell lines.

5. The device according to claim 1, wherein said therapeutic proteins have amino acid sequences which are identical to the amino acid sequences of naturally occurring proteins and/or are variants of naturally occurring proteins having an artificially constructed amino acid sequence.

6. The device according to claim 1, wherein at least one of said therapeutic proteins is a protein belonging to at least one of the protein families PDGF, VEGF, FGF, KGF, IGF, TGF, EGFL and/or variants of these proteins.

7. The device according to claim 1, wherein said therapeutic proteins are a combination of EGFL7, FGF-2, IGF-I, PDGF-B, TGF-β1 and VEGF-A, wherein one or more of these proteins may be substituted by one or more variant of the respective protein.

8. The device according to claim 1, wherein the therapeutic proteins are secreted in a concentration range of 5 pg/ml to 20 ng/ml for each of the therapeutic protein.

9. The device according to claim 1, wherein said container is a semipermeable hollow fiber, allowing the exchange of nutrients and the therapeutic proteins.

10. The device according to claim 1, wherein said container comprise semipermeable microcapsules, allowing the exchange of nutrients and the therapeutic proteins.

11. The device according to claim 1 for use in the treatment of wounds.

12. The device according to claim 1 for use in the promotion of angiogenesis.

13. The device according to claim 1 for use in the promotion of bone remodeling, peripheral artery disease, chronic artery disease, ischemia, organ repair after ischemic stroke, aortic/arterial wall injury, atherosclerosis, bone repair.

14. The device according to claim 1 for use in the treatment of conditions in veterinary medicine: osteohondroses, equine anovulatory haemorrhagic follicles (AHFs), equine deep stromal abscesses, equine vasculature anomalies, tibial dyschondroplasia.
